(19) United States
(12) Patent Application Publication
(10) Pub. No.: US 2025/0195277 A1
(43) Pub. Date: Jun. 19, 2025

(54) Ears protection device from water during washing of head or taking a shower and from heat when using hot hair-styling tools or hairdryer

(71) Applicant: Genady Mikhlin, Concord (CA)

(72) Inventor: Genady Mikhlin, Concord (CA)

(21) Appl. No.: 18/763,408

(22) Filed: Jul. 3, 2024

Related U.S. Application Data
(60) Provisional application No. 63/611,645, filed on Dec. 18, 2023.

(51) Int. Cl. A61F 11/14; A45D 44/12

(52) U.S. Cl. CPC A61F 11/14; A45D 44/12

(57) ABSTRACT

Disclosed in the present invention is an ears protection device from water during washing of head or taking a shower and from heat when using hot hair-styling tools or hairdryer. The device comprises cups made of silicone with drainage holes, connected via an arch made of nylon or other plastic material, designed to protect the ear from water during showering and from heat when using hot hair-styling tools or a hairdryer.

### TECHNICAL FIELD OF INVENTION

The present invention relates to safety and personal care of human ear, and more specifically, to a device comprising cups made of silicone with drainage holes, connected via an arch made of nylon or other plastic material, designed to protect the ear from water during showering and from heat when using hot hair-styling tools or a hairdryer.

### BACKGROUND OF THE INVENTION

When an individual is exposed to sudden changes in pressure, such as during airplane travel, scuba diving, or a direct blow to the ear, as well as exposure to loud noises, injury, or head trauma, their eardrums can become ruptured. Unfortunately, a ruptured eardrum may not always heal completely, which leads to various personal hygiene issues, such as water penetrating through the damaged eardrum into the middle ear during showering. Therefore, there is a need for a device that allows individuals to protect their ear from water. After conducting searches, purchasing, and testing commercially available devices, it was found that they had various shortcomings, rendering them ineffective in their primary function of water protection. As a result, despite using these devices, individuals still have to cover their ears with their hands and avoid exposing them to water streams. Consequently, there is a need to develop a device that addresses the shortcomings of existing models and provides effective ear protection.

In the course of studying existing ear protection devices, another common problem related to ear protection was identified: protection from high temperatures that ears are subjected to during certain hair care procedures, such as hair drying with a hairdryer or using specialized heating devices for curling or straightening hair. Commercially available devices for such protection have similar shortcomings as those found in devices for water protection. This justifies the creation of a device that effectively protects the ears from both water and high temperatures.

To begin with, it important to mention existing devices and their shortcomings. There are two groups of devices for protecting the ears from water and high temperatures:
The first group is of external applications, that are applied to the outer part of the ear and protect the ears either only from water or both from water and high temperatures.
The second group is devices for internal use that are installed inside the ear canal, and are serving only for protection from water.

### Examples of External-Use Devices.

Existing Device 1: The device is made in the form of plastic or silicone cups separate for each ear, so cups are not connected to each other. Such devices are used for protection both from water and high temperatures.

Shortcomings: Unreliable attachment of the cup to the ear, allowing it to easily come off the ear due to instability or under the pressure of water, leaving the ear completely unprotected. When using a hairdryer, the cups easily come off the ear under the pressure of the blown air. It is necessary to hold the cup with the hand and expose the hand to high temperatures.

Poor sealing of the cup edges to the head, leaving large gaps for water or hot air to penetrate. Some cups are made of overly thin material and provide inadequate protection from high temperatures. Plastic cups may melt upon contact with electrical devices such as a hair straightener or curling iron.

Existing Device 2: The device is made in the form of silicone cups connected to each other by a wire arc, which is rigidly attached to the cups. Such devices are used for protection from both water and high temperatures.

Shortcomings: Due to the poor shape of the cups and the rigid connection with the arc, it is practically impossible to position the cups in a way that they cover the ears well and are tightly pressed to the head. The wire arc in this device makes the structure rigid and difficult to adapt to the contours of the head. Additionally, people's ears not only have different sizes but also have different tilt angles. Because the cups have poor mobility, putting them on the ears is a very challenging task. As a result, significant gaps remain for water or hot air to penetrate.

Existing Device 3: The device is made in the form of a polyethylene bag worn on the ear and held in place by an elastic ring. Such devices are used only for protection from water.

Shortcomings: Although the bag is easy to put on the ear and secure by a rubber ring behind the ear, this does not guarantee effective protection from water penetration. Since the ear has a complex and convoluted shape with open areas at different levels, it is not easy to cover all these opening by bag, leaving entrances to the water. Additionally, the ear is a soft cartilaginous organ, and any attachment to it will be unreliable. Therefore, under a stream of water, the bag can be knocked off the ear. Also, during time spent under the shower, water gradually penetrates inside the bag, accumulating there, and upon reaching a certain amount it flows directly into the ear. The bags are disposable and require replacement after each use.

Existing Device 4: The device is made in the form of a polyethylene film that has adhesive strip around the perimeter which adheres to the ear. Such devices are used only for protection from water.

Shortcomings: Unreliable adhesion to the ear and leaving gaps for water penetration. Use of adhesive, which can be an irritant to the skin. Single-use device.

Existing Device 5: These are example of devices of internal use. Devices are inserted into the ear canal, such as silicone and paraffin earplugs. Such devices are used only for protection from water. Existing device is made as silicone earplugs or wax earplugs.

Shortcomings: Unreliable installation in the ear, leading to insufficient water protection. Possibility of the device falling out of the ear under the pressure of water, leaving the ear unprotected. The device comes into contact with earwax, requiring either special care or replacement. For individuals with ear conditions, the process of inserting the device can be painful.

In short, the above background art has revealed that there is a variety of ear protectors in the market. However, there are a few prior art documents that describes an ear protection device from water during washing of head or taking a shower and from heat when using hot hair-styling tools or hairdryer as proposed by the client.

The proposed device is designed to protect the ears of human beings from water entering the ear canal as well as from the impact of high temperatures when using hair care electrical devices. The device consists of two oval-shaped cups made of high-strength silicone, and an arc, which has the shape of an open circle and is made of nylon. The arc connected to the cups through special ball-joint connectors.

Therefore, introduction of a new device for protecting the ears comprising features being proposed in the present invention is dire.

### OBJECTS OF THE INVENTION

The main object of the present invention is to provide a safety and personal care of human ear device made of plastic and silicone.

The other object is to provide a device designed for protection of the ear from water during washing of head or taking a shower and from heat when using hot hair-styling tools or hairdryer.

The following summary is an explanation of some of the general inventive steps for the system, method, architecture and container in the description.

### SUMMARY OF THE INVENTION

The following is a summary of the invention, which is not an extensive overview of the invention. This summary is not intended to identify critical elements of the invention or in any way to delineate the scope.

In one general embodiment, the present invention describes an ear protection device from water during washing of head or taking a shower and from heat when using hot hair-styling tools or hairdryer as proposed by the client.

In another preferred embodiment, the device is designed to protect the ears of human beings from water entering the ear canal as well as from the impact of high temperatures when using hair care electrical devices.

In another embodiment, the device consists of two oval-shaped cups made of high-strength silicone, and an arc, which has the shape of an open circle and is made of nylon. The arc connected to the cups through special ball-joint connectors.

In another preferred embodiment, the device can be used while taking shower, drying hair with hairdryer and while styling hair with hair straightener.

Yet in another embodiment, the device demonstrates an arc connected to the cups bearing drainage holes through special ball-joint connectors.

### BRIEF DESCRIPTION OF DRAWINGS

The novel features believed to be characteristic of the illustrative embodiments are outlined in the appended figure and claims. The illustrative embodiments, however, as well as a preferred mode of use, further objectives, and descriptions thereof, will best be understood by reference to the following detailed description of one or more illustrative embodiments of the present disclosure when read in conjunction with the accompanying drawing.

FIG. 1 illustrates an assembled device, when not worn on the head.

FIG. 2 is a side view when my device worn on the head with arch under chin.

FIG. 3 is a side view when my device worn on the head with arch behind the neck.

FIG. 4 shows the arch securely joins with ear cups and constantly applies pressure to the center of each ear cup through the ball joints.

FIG. 5 is the ball joint evenly spread arch pressure to the rims of ear cup, making ear cup to adjust to surface of the head.

FIG. 6 illustrates the drainage hole prevents seeping water from gathering and entering ear canal.

FIG. 7 is the drainage holes on the bottom of ear cup.

FIG. 8 shows when the arch is widened it exerts pressure due to resistance of material.

The above figures describe an automated smoothie dispensing system and components contained in therein, their layout, and location. They only identify the possible various tools and location of the compartment. Customization of the device to a specific model is possible.

### DETAILED DESCRIPTION OF INVENTION

In the following detailed description of the invention, numerous details, examples, and embodiments of the invention are described. However, it will be clear and apparent to those knowledgeable in the field that the invention is not limited to the embodiments set forth and that the invention can be adapted for a wider range of applications.

In this description, the terms “comprising,” “including,” “containing,” “characterized by,” and grammatical equivalents thereof are inclusive or open-ended terms that do not exclude additional, un-recited elements or method acts, but also include the more restrictive terms “consisting of” and “consisting essentially of” and grammatical equivalents thereof. As used herein, the term “may” with respect to a material, structure, feature or method act indicates that such is contemplated for use in implementation of an embodiment of the disclosure and such term is used in preference to the more restrictive term “is” so as to avoid any implication that other, compatible materials, structures, features and methods usable in combination therewith should or must be, excluded.

The singular forms “a,” “an,” and “the” are intended to include the plural forms as well, unless the context clearly indicates otherwise.

The term “and/or” includes any and all combinations of one or more of the associated listed items. As used herein, relational terms, such as “first,” “second,” “top,” “bottom,” “upper,” “lower,” “over,” “under,” etc., are used for clarity and convenience in understanding the disclosure and do not connote or depend on any specific preference, orientation, or order, except where the context clearly indicates otherwise.

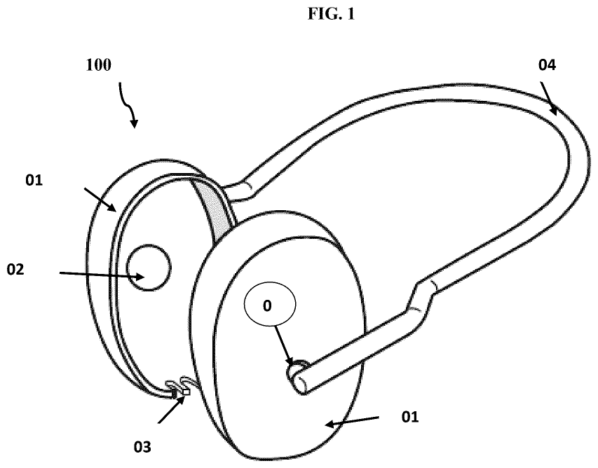

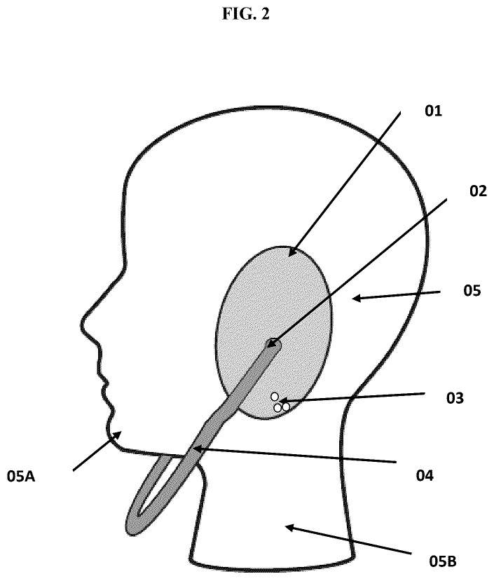

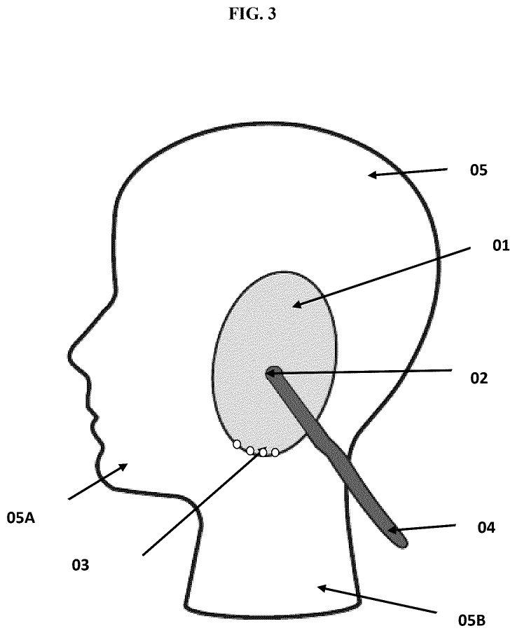

Turning to Figures, FIG. 1 illustrates an assembled device 100, when not worn on the head. The device 100 has an ear cup 01 connected to arch 04 that terminates in a ball joint 02. The car cup has small drainage holes 03. In FIG. 2, the invention illustrates a side view of the device 100 when it is worn on the head 05 with arch 04 under chin 05A. In FIG. 3, invention is a side view of the device 100 worn on the head 05 with arch 04 behind the neck 05B. It also illustrates the location of the drainage holes 03, ball joint 02 and ear cup 01.

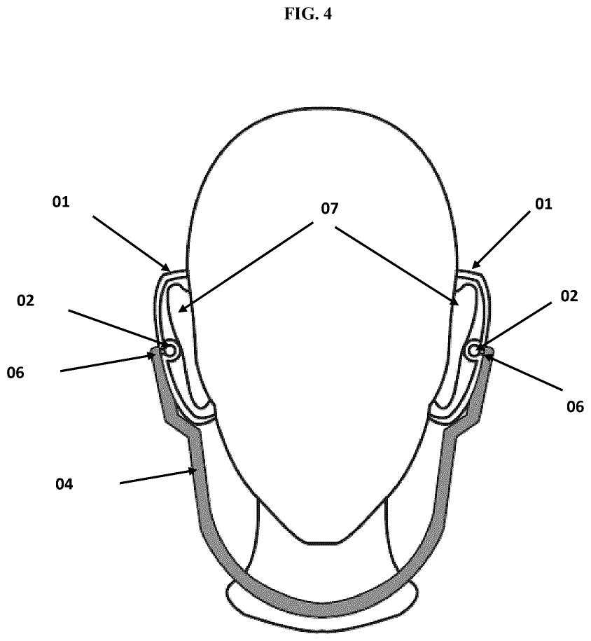

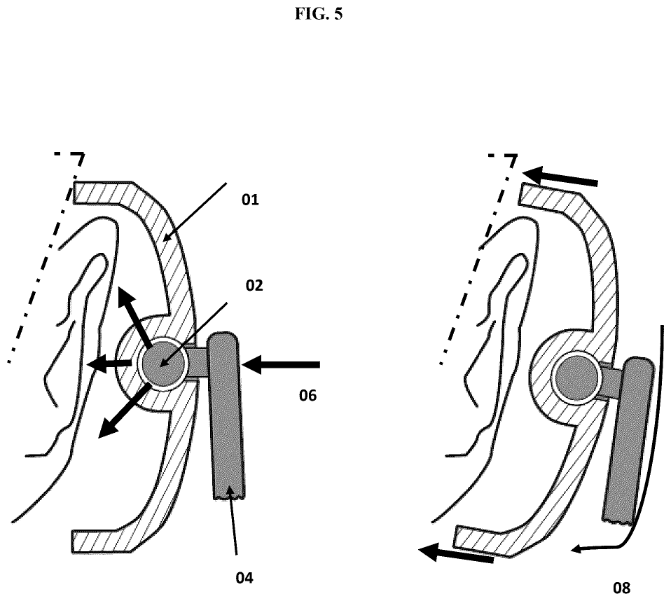

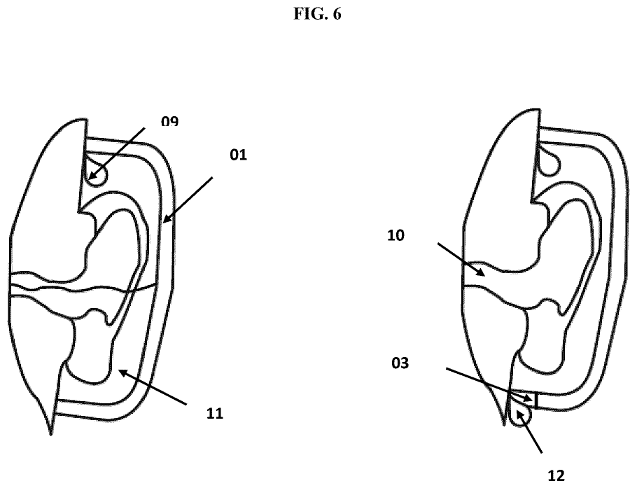

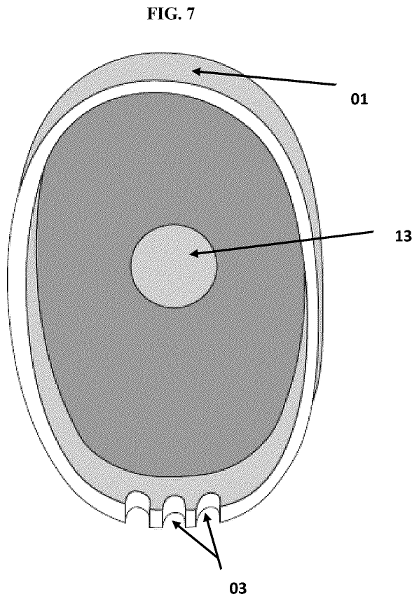

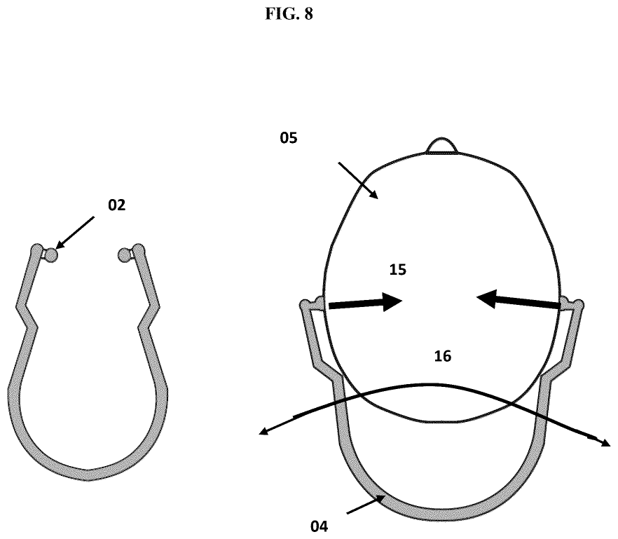

In FIG. 4, the invention shows how the arch 04 securely joins with ear cups 01 and constantly applies pressure 06 to the center of each ear cup 01 through the ball joints 02 of the human ear 07. In FIG. 5, the ball joint 02 evenly spread arch pressure 06 to the rims of ear cup 01, making ear cup 01 to rotate at position 08 and adjust to surface of the head 05. FIG. 6 illustrates how the drainage hole 03 prevents seeping water 09 from gathering and entering ear canal 10. It also shows escaping water 12 from the cup 01. In FIG. 7, the invention shows the drainage holes 03 on the bottom of ear cup 01 and the ball joint socket 13. FIG. 8 shows when the arch 04 is widened at position 16 to exert pressure to exert pressure 15 through ball joints 02 due to resistance of material.

The detailed description below, in conjunction with the accompanying drawings, serves as an illustrative description of examples and it not meant to be the exclusive representation of the various forms in which the present examples can be created or utilized.

### Description of the Invention (Device)

The described device can be classified as an external-use device, protecting the ears 07 from the outer side of the ear according to FIG. 4. It is designed to protect the ears from water 09 entering the ear canal 10 as well as from the impact of high temperatures when using hair care electrical devices as taught in FIG. 6.

The foundation of the device 100 is based on the following key principles that the device should consist of two cups 01 with drainage holes 03 connected by an arch 04. The connection of the arch 04 and cups should allow the cup 01 to remain movable, capable of rotating around vertical 08, horizontal axes, and an axis perpendicular to the main plane of the cup 01, which allows the cups to be oriented to the tilt of the ear 07 and fully cover the ears, adapting to the contours of the head 05 as shown in FIG. 5. The arch 04 should exert pressure 06 on the cups so that they can assume the most optimal position on the head 05 to ensures that the cups 01 snugly adhere to the head's surface, providing better resistance to water or hot air penetration and secure retention of the cups in the adopted position against displacement caused by water or air pressure or head movements. Cups should have cutouts for water drainage, preventing accumulation of water in case of accidental penetration of small amounts of water under the cup 01 and subsequent entering of water into the ear canal 10 as shown in FIG. 6.

While the above principles are incorporated in the present device 100, it is possible to have variations devices built on these principles.

The device consists of two oval-shaped cups 01 made of high-strength, heat resistant silicone, and an arc, which has the shape of an open circle and is made of nylon. The arch 04 is connected to the cups through special ball-joint connectors 02. In the assembled state, when not worn on the head, the device looks like as assembled in FIG. 1. FIG. 2 and FIG. 3 show how the device looks when worn on the head with arch under chin and behind the neck.

### How the Invention (Device) Addresses the Problems in the Prior Art

In reviewing several typical drawbacks of existing devices and how the present device addresses these issues.

Drawback 1: Unreliable fastening of the cup to the ear. The solution as provided in the present invention is that the device has cups connected by an arch 04 that holds them together and prevents them from falling. See FIG. 4. The cups 01 can freely rotate on the tip of the arch 04, thanks to a special ball-and-socket joint 02, allowing them to easily adapt to the shape of any ear 07 and find a position where the cups fully cover the ears. The arch 04 constantly applies pressure to the cups, pressing them against the head 05. This provides an additional guarantee that the cups 01 will not be dislodged by water, air, or head movements. See FIG. 4. The size of the cups is experimentally chosen to fit the dimensions of an adult's ears 07. The shape of the cup 01 rims does not have complex bends, ensuring a secure coverage of the ear 07. Ear cups can rotate 08 around its own axes to fit ear angle tilt as taught in FIG. 5.

Drawback 2: Poor sealing of the edges of the cup rims to the head 05, leaving large gaps for water or hot air penetration. The solution as taught in the present invention is addressed by using a ball-and-socket joint 02 between the arch 04 and the cup 01. This connection allows the cup freedom of movement in space. With this joint, when the arc presses on the cup 01, the cup rotates, adapting to the contour of the head and tightly pressing against the head 05 for stability and resistance to pressure 06. Sec FIG. 5.

Additionally, the cup 01 has a specially simplified oval shape with straight rims, ensuring that the shape of the ear 07 does not hinder the cup 01 from properly positioning itself on the head 05. This adapted position and the snug fit of the cup almost completely eliminate gaps between the cup rims and the surface of the head 05, creating a barrier against water or hot air. FIG. 5 shows a ball joint 02 evenly spread arch pressure to the rims of ear cup 01, making ear cup to adjust to surface of the head 05.

Drawback 3: Water seeping 09 into the cup accumulates in the cup and flows inside the ear 07. The solution lies in the fact that due to the unevenness of the head 05, it is practically impossible to prevent water from seeping 09 under the cup 01 entirely. In the device 100, thanks to a tighter and more uniform fit of the cup to the head's surface, water seeping is minimized. The water that does manage to seep under the cup 01 is essentially individual droplets that flow down the skin of the head 05. As these water droplets flow downward 12, they reach the lower part of the cup 01 and, thanks to drainage holes 03, exit to the outside instead of accumulating at the bottom of the cup 01. Since the entrance to the ear canal 10 is at some distance from the head's surface, water droplets 12 cannot penetrate inside the ear canal 10. Thus, the internal part of the ear canal 10 remains dry. See FIG. 6 which shows how seeped water 09 expelled through drainage holes 03 preventing from gathering 11 and entering ear canal 10.

Drawback 4: Some cups are made of overly thin material and provide poor protection against high temperatures. The solution provided is that the cups 01 in the device 100 are made of 3 mm silicone, which is resistant to high temperatures up to 300 degrees Celsius (meaning it will not melt upon contact with a hot device) and serves as a good thermal insulator. Additionally, the cups 01 have sufficient depth to create an air gap between the ears 07 and the cup walls. This design further enhances thermal insulation.

Drawback 5: For people with ear conditions, the process of inserting internal ear protection device can be painful. The device 100 is designed for external use, so even individuals with ear conditions can comfortably wear it since it does not interfere with the inner ear. Furthermore, individuals using hearing aids can use the device 100 without removing their hearing aids.

### Description of Preferred Embodiments

The following is a more detailed information on the development of each element of the device, outlining the requirement set and their solutions.

### The Ear Cup

The first element of the device 100 to be considered is the ear cup 01. Requirements for the ear cup's design and their solutions. Requirement for the cup 1: The cup 01 should maximally cover the ear 07 to ensure reliable ear protection. Solution resides in the following; firstly, since the shapes of people's ears vary greatly. The cup 01 is not made in the shape of an ear 01 but designed as an oval. Selected the size of the oval to cover the majority of average-sized adult ears. The internal dimensions of the cup's oval are 75 mm+/−3 mm for the longer part and 45 mm+/−3 mm for the shorter part. These dimensions are sufficient to cover the sizes of ear 07 for the majority of people while keeping ear cups 01 compact.

Although, if the cups are slightly larger, it will still work. Additionally, once the device 100 is assembled, the cup retains its freedom to rotate around its axis. This allows the cup to align with the ear's angle of inclination and reliably cover it completely. See FIG. 6.

Requirement for the cup 2: The cup 01 should sit securely and should not fall by itself or come off if a strong stream of water or air is directed at it. The solution is to ensure that the cup sits securely on the ear 07, it is connected to the arch 04 through a special ball-joint 02. The arch 04 has a spring force that constantly presses on the cup, thereby keeping it in the adapted position. The two cups connected through the arch 04 transfer the pressure from the arch 04 towards each other, adding stability to the structure. Therefore, the cups will sit securely even when standing under a strong shower or in the stream of air from a hairdryer, or when turning or tilting the head 05. Sec FIG. 4.

Requirement for the cup 3: The cup should snugly fit the head 05, following the contours of the head, to minimize gaps and, consequently, the possibility of water or hot air penetration. Solution: To achieve this, the unique ball-joint connection 02 between arch 04 and cups 01 is used in device 100.

A ball joint 02 for the design has other connections that allow the cup 01 to rotate freely in space, which can also be used. So, the ball joint provides the necessary level of freedom for the cup to orient in space and snugly adhere to the head 05, following its contours.

Secondly, for the cup 01 to orient in space, following the contours of the head 05 independently, the cup needs to be subjected to pressure. Only under pressure will the cup 01, having mobility, begin to rotate and assume the required position. The arch 04 generates this pressure due to its springiness. In the free state, the distance between the hinge ends of the arch is much smaller than the diameter of the head. Therefore, when putting on the device, the arch 04 must be widened to a size exceeding the head's diameter. By widening the arch, the device 100 is forced to bee pressed on the cups due to its resistance 15 as in FIG. 8.

Thirdly: thanks to the mobility of the cup, it can be oriented in such a way that it correctly covers the entire ear 07. Since people's ears not only have different sizes but also inclinations, this ability is also crucial. The preferred design is a ball joint. See FIG. 5.

Requirement for the cup 4: The cup should be relatively mobile so that, when turning the head, the cup 01 can change the contact angle, thereby preventing the formation of large gaps between the head 05 and the cup through which water or air can enter.

Solution: This is achieved by the following features of the device 100: Proper position of the cups 01 on the head 05 surface reducing water penetration under cup 01 and drainage holes 03 located on the cup 01 to prevent water accumulation and entering into ear 07.

Requirement for the cup 5: The cup 01 should prevent water from entering the ear 07 and have the ability to get rid of seeping water 09 so that it does not accumulate and flow into the ear 07.

Solution: This section outlines another feature of the device 100. At the very beginning of working on the device, the issue was how to prevent water from getting under the cup 01 altogether. Trying out many different options, it was realized that this task is almost impossible given the unevenness of the head, different ear 07 configurations, and so on. Then there was need to reformulate the task, namely, to prevent water from getting inside the ear 07.

The present device 100 copes with this task perfectly. How? The main task of the cup 01 is to take on the shower streams and dampen the pressure of the stream and splashes. Thus, in places where the cup fits tightly to the head, water is completely blocked, in places where there are minimal clearances, water can seep inside 09, but it does so without splashes and pressure. That is, such water cannot get into the ear canal, only seeping along the surface of the head 05. Thus, an additional task is not to allow this water to accumulate, since then its level will rise, and it will enter into the ear canal 10.

To prevent water from accumulating, drainage holes 03 were added to the cup's design, which, when the device 100 is worn correctly, should be at the bottom of the cup 01 after several experiments. These should be several small drainage holes 03. Thanks to the small size of the holes, they will not be covered by the soft skin of the check. If there were one large hole instead of small ones 03, then the soft skin of the check would bend and fill that hole. Another advantage of small holes is that if a person raises their head 05 and the water streams can reach the holes, due to their small size, these holes are not sufficient for the lateral streams of water to penetrate. See FIG. 7 which shows drainage holes on the bottom of ear cup 01.

Requirement for the cup 6: The cup 01 should be made of a material that is resistant to water and high temperatures, and is considered a good thermal insulator.

Solution: Since the cup will be exposed to water and high temperatures, it must be made of a material that is resistant to these influences. Therefore, silicone was chosen for the cups 01 as in FIG. 8. It has good water resistance and can withstand temperatures up to 300 degrees Celsius.

Requirement for the cup 01: The walls of the cup 01 should not fit tightly against the ear 07 but have a small air gap. This effectively protects the ear from high temperatures.

Solution: As my device should protect the ears 07 from the impact of high temperatures, the design includes an air gap between the surface of the cup 01 and the ear itself, as air is a good thermal insulator. There is an air gap between ears and walls of ear cup 01.

Requirement for the cup 01: The cup 01 should be made of a material that is considered safe for human skin.

Solution: Since the cup 01 comes into contact with human skin, it must be made of a material that is safe for human skin. The chosen silicone also has this property and is safe for humans.

Requirement for the cup 9: The design and material of the cup 01 must be such that the cup does not deform under the pressure applied by the arch 04.

Solution: As mentioned earlier, the cups experience constant pressure from the arch 04. This pressure is transferred from the point where the cup 01 is connected to the arch 04 to the edges of the cup that are in contact with the head 05. To effectively transmit this pressure without deformation, the cup must be resistant to deformation. To prevent deformation, the cup 01 is made of high-strength silicone.

### The Arch

The second main element of my device is the arch 04. The arc serves the following purposes: Firstly, it connects to the cups 01, forming a unified object and allowing the cups to be more securely held on the head 05. Secondly, it transmits pressure to the cups, enabling them to orient themselves correctly in space and settle with minimal gaps on the head.

Requirement for the arc 1: The arch 04 should attach to the cups 01 without restricting their mobility.

Solution: This was achieved through a ball-and-socket joint 02 type of connection. The ends of the arch 04 have a spherical shape, while the cups 01 have a spherical recess. Inserting the spherical end into the recess creates a ball-and-socket joint. See FIG. 5.

Requirement for the arc 2: The arch 04 should exert pressure on the ear cups 01.

Solution: The arch 04 is shaped like an incomplete circle. The distance between the ends of the arch 04 is smaller than the diameter of a person's head 05. To wear the device 100 on the head, the arc needs to be widened to a distance sufficient to seat the cups on the ears 07. Since the arch 04 is made of a material that attempts to regain its shape after deformation, the stretched arch creates the necessary pressure, due to its resistance. See FIG. 8, which shows that when arch 04 is widened it exerts pressure due to resistance of material.

Requirement for the arch 3: The arc should be durable and not break with repeated use or bending slightly beyond the required bend.

Solution: As the arc constantly experiences bending and stretching, it must be made of a material that reliably withstands such loads. Nylon was chosen as the material because it is much stronger than plastic and more resistant 15 to bending stresses.

Requirement for the arc 4: The arch 04 should have a shape and design that minimally obstructs a person from performing procedures in the head area while washing the head 05, drying and styling hair, washing the face.

Solution: The dimensions of the arch 04 were chosen to be unobtrusive and not conceal parts of the head 05 during these procedures. Therefore, the arch 04 is shaped like a thin, curved rod around the head, either under the chin or at the back of the neck 05A, practically not hindering procedures in the head area.

Requirement for the arc 5: The arch 04 should not irritate the skin at points of contact.

Solution: To achieve this goal, the arch 04 has a very smooth surface and is made of a material that does not irritate the skin-nylon.

### Assembled Device

Requirement for the entire device 1: Easy to assemble and disassemble.

Solution: Since the device 100 consists of cups and an arch 04, it needs to be assembled before use. Assembling the device is straightforward: bring the spherical end of the arch 04 to the hole in the cup 01 and insert the end of the arch 04 into the hole. Since the cup 01 is made of silicone, inserting the end of the arch 04 into it is effortless. If disassembly is required, hold the cup 01 in one hand and pull the end of the arc out of the cup's hole. The procedure is performed without much effort.

Requirement for the entire device 2: Easy to put on and take off.

Solution: To put on the assembled device 100, hold it by the sides of the cups 01. Make sure the drainage holes 03 of the cups are facing downward. While holding the cups 01 in the hands, spread them apart to a distance sufficient to place the cups on the ears 07. Make sure the cups 01 are securely in place and completely cover the ears 07.

Recommended two positions for the device: First, the arch 04 is positioned under the chin. See FIG. 2. Second, the arch 04 is positioned at the back of the neck 05A. To remove the device 100, hold it either by the sides of the cups or by the tips of the arch 04. Gently stretch them to easily remove them from the ears and take them off.

Requirement for the entire device 3: Securely stay on the head and perform protective functions.

Solution: The described construction, thanks to the cups 01 which securely covering the cars 07, being connected to the resilient arch 04 through hinges, and being pressed against the head by this arch 04, allows the device to securely stay on the head 05, not falling off during head movements or under the impact of water streams or air blow, and remaining constantly tightly pressed to the head 05, thereby maximizing the blockage of water or air penetration.

Requirement for the entire device 4: Easy to store.

Solution: The device requires no special care for storage. Just let it dry after using under shower.

Requirement for the entire device 5: Easy to carry for trip.

Solution: To carry for trip, device 100 can be simply placed in an assembled state if there is enough space. If space is limited, the device can be disassembled and put in the plastic bag to take smaller space.

Requirement for the entire device 6: Reusable.

Solution: The materials used to make the device 100 are durable and designed for multiple uses.

### Examples of Intended Use

The invention has examples of the application of my device including when the user is taking a shower while using the device 100, when drying hair with the device, using my device while drying hair with hairdryer and curling hair with the device or using the device while styling hair with hair straightener.

### Advantages of the Device

a. Effectively protects ears 07 from water entering the ear canal while taking a shower or washing the head 05;
b. Effectively shields ears 07 from the impact of high temperatures when using hot hair care devices;
c. Suitable for use by most adult individuals of any gender.
d. Easy to put on and take off.
e. Can be used by individuals with ear conditions, hearing aids, or those highly sensitive to water entering the ears 07, and
f. Reusable, durable, and resistant to breakage.

While the invention has been described with reference to numerous specific sensors, one of ordinary knowledge in the field will recognize that the invention can be embodied in other specific forms without departing from the spirit of the invention. Specific operations may not be performed in one continuous series of operations, and different specific operations may be performed in different embodiments.

Furthermore, the processes or methods could be implemented using several sub-processes, or as part of a larger macro process. Thus, one of ordinary knowledge in the field would understand that the invention is not to be limited by the foregoing illustrative details, but rather is to be defined by the appended claims.

### INDUSTRIAL APPLICATION

The present invention applies to the safety and personal care industry. It provides a device made of plastic and silicone and designed for protection of the ear from water during washing of head or taking a shower and from heat when using hot hair-styling tools or hairdryer. Main advantages of my device include being effectively in protecting ears from water entering the ear canal while taking a shower or washing the head and in protecting from heat while doing hairstyling with hot tools. Examples of the application of my device include when the user is taking a shower while using the device, using my device while drying hair with hairdryer, or curling hair with the curler or using the device while styling hair with hair straightener.

What is claimed is:

1. A device for protection of the ear from water during washing of head or taking a shower and from heat when using hot hair-styling tools or hairdryer, comprising:
two oval-shaped cups made of high-strength silicone;
an arch, which has the shape of an open circle and is made of nylon, and
small drainage holes at the bottom of the cup.

2. The device according to claim 1, wherein the arc is connected to the cups through special ball-joint connectors.

3. The device according to claim 1, wherein the arc is under the chin or behind the neck.

4. The device according to claim 1, wherein the several small drainage holes are introduced at the bottom of the cup to ensure that the accumulated water drops off.

5. The device according to claim 1, wherein the device is designed to protect:
human from water during washing of head or taking a shower and from heat when using hot hair-styling tools or hairdryer as proposed by the client;
the ears of human beings from water entering the ear canal as well as from the impact of high temperatures when using hair care electrical devices, and
the user while taking shower, drying hair with hairdryer and while styling hair with hair straightener.

6. A device for protection of the ear from water and heat, comprising:
two oval ear cups;
an arch, and
small drainage holes at the bottom of the cup.

7. The device according to claim 6, wherein the oval cups are designed to cover the ear maximally and ensure reliable ear protection, and comprises internal dimensions of the cup's oval are 75 mm+/−3 mm for the longer part and 45 mm+/−3 mm for the shorter part to cover all sizes of ears of people while keeping ear cups compact and to ensure that once the device is assembled, the cup retains its freedom to rotate around its axis.

8. The device according to claim 6, wherein each cup is connected:
to the arc through a special ball-joint to ensure that the cup sits securely on the ear, wherein the arch has a spring force that constantly presses on the cup, thereby keeping it in the adapted position;
through the arch transfer the pressure from the arch towards each other, adding stability to the structure to ensure that the cups sit securely even when standing under a strong shower or in the stream of air from a hairdryer, or when turning or tilting the head, and
to the arc snugly fit the head, following the contours of the head, to minimize gaps so as to avoid the possibility of water or hot air penetration.

9. The device according to claim 8, wherein the ball joint for the design has other connections that allow the cup to rotate freely in space, which can also be used, wherein the ball joint provides the necessary level of freedom for the cup to orient in space and snugly adhere to the head, following its contours.

10. The device according to claim 9, wherein:
the arch is widened to ensure that the device is forced to be pressed on the cups due to its resistance;
the cup is relatively mobile so that, when turning the head, the cup can change the contact angle, thereby preventing the formation of large gaps between the head and the cup through which water or air can enter;
the connection between the cup and the arch ensures that the cups are constantly under pressure, when turning the head and making slight shifts, the cups always try to turn to snugly adhere to the head, thereby compensating for the pressure from the arch and preventing the formation of large gaps between the head and the cup;
the cup has small drainage holes at the bottom of the cup to ensure that if users raises their head and the water streams can reach the holes, due to their small size at the bottom of ear cup;
the cup is made of a silicone material because it is resistant to water and high temperatures, considered to be a good thermal insulator and can withstand temperatures up to 300 degrees Celsius, and
there is a small air gap between ears and walls of ear cup to effectively protect the ear from high temperatures.

11. The device according to claim 6, wherein the arc connects to the cups, forming a unified object and allowing the cups to be more securely held on the head and transmits pressure to the cups, enabling them to orient themselves correctly in space and settle with minimal gaps on the head.

12. The device according to claim 11, wherein the arc is attached to the cups through a ball-and-socket joint type of connection and ends of the arc have a spherical shape, while the cups have a spherical recess, and is fixed by inserting the spherical end into the recess creates a ball-and-socket joint.

13. The device according to claim 12, wherein the arc is made of nylon material that is durable and which does not break with repeated use or bending slightly beyond the required bend.

14. The device according to claim 13, wherein the arc is shaped like a thin, curved rod around the head, either under the chin or at the back of the neck, practically not hindering procedures in the head area.

15. The device according to claim 14, wherein the arc has a very smooth surface and is made of a material that does not irritate the skin-nylon.
